(19) United States
(12) Patent Application Publication
(10) Pub. No.: US 2011/0110849 A1
(43) Pub. Date: May 12, 2011

(54) METHOD OF CONVERTING A RAW MATERIAL STREAM INTO A PRODUCT STREAM USING A FLUIDIZED BED AND APPARATUS FOR USE IN SAID METHOD

(76) Inventor: Roland Siemons, Enschede (NL)

(21) Appl. No.: 12/990,321

(22) PCT Filed: Apr. 21, 2009
(86) PCT No.: PCT/EP2009/054750

§ 371 Date: Jan. 13, 2011

(30) Foreign Application Priority Data
Apr. 29, 2008 (EP) 08155330.7

(51) Int. Cl. B01J 8/18; C07C 5/327; C01B 3/26

(52) U.S. Cl. 423/655; 422/139; 422/141; 585/654

(57) ABSTRACT

The present invention relates to a method of converting a raw material stream into a product stream by passing said raw material stream through a bed of fluidized solid particles and allowing heat exchange to occur between the bed of fluidized solid particles and the raw material stream, said method being characterized in that it alternately employs a production mode and a restoration mode to control the temperature of the bed of fluidized solid particles: -said production mode comprising passing the raw material stream through the bed of fluidized solid particles and allowing the temperature of the fluidized solid particles in the bed to decrease or to increase as a result of the production energy associated with the conversion of the raw materials stream into the product stream; and -said restoration mode comprising restoring the temperature of the bed of fluidized solid particles by passing a restoration stream through the bed of fluidized solid particles to decrease the temperature of the fluidized solid particles in case the temperature of the fluidized solid particles has increased during the production mode or to increase the temperature of the fluidized solid particles in case the temperature of the fluidized solid particles has decreased during the production mode, and that -the fluidized solid particles are kept in the same reactor. The present method enables continuous operation of the fluidized bed reactor without the need of recirculating externally heated or cooled solid bed particles and/or for special heat exchange equipment placed inside or enclosing the fluidized bed reactor.

### TECHNICAL FIELD OF THE INVENTION

The present invention relates to a method and apparatus for making a product using at least one intermittently operated fluidized bed.

### BACKGROUND OF THE INVENTION

Fluidized bed reactors are usually vessels that are filled with particles of one or more types of solid matter through which gas or liquid streams are flown to bring the particles into a state of fluidization. Fluidized bed reactors are usually designed for carrying out a chemical or physical process within the fluidized bed under substantially stationary physical and/or chemical conditions.

In order to maintain the desired stationary physical and chemical conditions in the fluidized bed reactor it is often necessary to regularly, if not constantly, remove or add solid material and/or energy from/to the fluidized bed reactor.

Examples of such processes were known from e.g. GB 789228A; GB 832143A; EP-A-0235531; EP-A-0765849 and U.S. Pat. No. 2,667,448.

For the removal and/or addition of solids and/or energy to the fluidized bed reactor, specific facilities, such as conveyor means, cyclones, heat exchangers, and pressure adjusting means are needed and sometimes these facilities need to be integrated with the fluidized bed reactor. Such specific facilities are relatively expensive to install and/or to exploit and may require maintenance and/or repair because of their vulnerable character.

It is known in the art to introduce heat into the fluidized bed of a reactor in order to ensure that the thermal conditions within the bed remain constant over time. One particular option for providing energy to a stationary fluidized bed, is the use of an internal burner which releases combustion products directly into the fluidized bed. These combustion products, however, mix with the primary reacting product and/or reactant. Thus, there is a risk of contamination and/or reduced product yield. In addition, this particular set-up has the disadvantage that it may necessitate separation of reactants and/or reaction products from the combustion products.

It is further noted that particular peripheral facilities sometimes are not technically effective in maintaining stationary conditions within the fluidized bed reactor. For instance, heat transfer through the reactor wall may be rate limiting and interfere with a scaling up beyond limits that are determined by the ratio of the outer reactor surface and the reactor volume of the fluidized bed reactor.

The present invention provides a method and apparatus for making a product using at least one fluidized bed reactor which avoid and/or minimize the above mentioned drawbacks and/or disadvantages.

### SUMMARY OF THE INVENTION

The present invention provides a method of converting a raw material stream into a product stream by passing said raw material stream through a bed of fluidized solid particles and allowing heat exchange to occur between the bed of fluidized solid particles and the raw material stream, said method being characterized in that it alternately employs a production mode and a restoration mode to control the temperature of the bed of fluidized solid particles:
said production mode comprising passing the raw material stream through the bed of fluidized solid particles and allowing the temperature of the fluidized solid particles to decrease or to increase as a result of the production energy associated with the conversion of the raw material stream into the product stream, and
said restoration mode comprising restoring the temperature of the bed of fluidized solid particles by passing a restoration stream through the bed of fluidized solid particles to decrease the temperature of the fluidized solid particles in case the temperature of the fluidized solid particles has increased during the production mode or to increase the temperature of the fluidized solid particles in case the temperature of the fluidized solid particles has decreased during the production mode;
and that the fluidized solid particles are kept in the same reactor.

The invention is based on the following insights:
1) by operating a fluidized bed reactor in a production mode without maintaining the physical and/or chemical conditions within the fluidized bed reactor stationary, production conditions will only gradually (as opposed to abruptly) change to a level at which the production will proceed no longer or at an unpractical or non-economical rate;
2) this gradual change of production conditions is often due to gradual energy exchange with the fluidized solid particles, which particles as a result gradually loose their capability of absorbing or releasing production energy;
3) the capability of fluidized solid particles to absorb or release production energy can be restored by passing a restoration stream through the fluidized bed that causes cooling or heating of the fluidized solid particles to an adequate level;
4) changes in production conditions can often be tolerated within a relatively broad temperature window;
5) significant advantages can be realised by operating a fluidized bed reactor in a production mode without maintaining constant physical and/or chemical conditions, provided the latter conditions are restored during a subsequent restoration mode before production yields and/or product quality reach unacceptable levels;
6) the aforementioned production mode and restoration mode can be operated alternately in a continuous fashion for an indeterminate period of time;
7) the restoration mode, which is defined relative to the production mode that yields the primary product stream, can be designed in such a way that it also yields a useful product, meaning that both the production mode and the restoration mode are de facto productive modes.

An important advantage of the present invention resides in the fact that external specific facilities for maintaining physical and/or chemical process conditions within the fluidized bed reactor, and for the provision or removal of process energy are no longer required. More particularly, the present method enables continual operation of the fluidized bed reactor without the need of special equipment for the recirculation of externally heated or cooled solid particles, and without the need of heat exchangers placed inside or enclosing the fluidized bed.

It will be appreciated that the process of the present invention, using an intermittent fluidized bed, does solve the particular problems connected to operating a fluidized bed in a continuous manner (prior art process).

These problems are:
the need to regularly, if not constantly remove or add solid material and/or energy from/to the fluidized bed reactor,
the need for an additional solids restoration or conditioning reactor to and from where the solids are to be transferred.
the need for specific devices such as conveyor means, cyclones, heat exchangers and pressure adjusting means,
the need for means to avoid mixing of reactants and regeneration gases.
the suitability of appendages for high temperatures,
wear resistance of appendages,
energy consumption of appendages, and
complex process controls.

### BRIEF DESCRIPTION OF THE DRAWINGS

FIG. 1 is a schematic view of a fluidized bed reactor which can be operated intermittently in a production mode and in a restoration mode.

FIG. 2 shows a schematic view of two coupled fluidized bed reactors that are individually intermittently operated in a coordinated manner with each other (i.e. in counter-phase), and wherein the two operational modes are both productive, and, hence, each production mode is the restoration mode of the other production mode.

FIG. 3 shows a schematic view of a prior art process using stationary fluidized beds from which solid material and/or energy is regularly removed.

FIG. 4 shows a schematic view of a process according to the present invention, using intermittently operated fluidized beds and comprising a pyrolytic liquefaction of biomass (production mode) and a combustion reaction (restoration mode).

### DETAILED DESCRIPTION OF THE INVENTION

Accordingly, one aspect of the invention relates to a method of converting a raw material stream into a product stream by passing said raw material stream through a bed of fluidized solid particles and allowing heat exchange to occur between the fluidized solid particles and the raw material stream, said method being characterized in that it alternately employs a production mode and a restoration mode to control the temperature of the bed of fluidized solid particles:
said production mode comprising passing the raw material stream through the bed of fluidized solid particles and allowing the temperature of the fluidized solid particles to decrease or to increase as a result of the production energy associated with the conversion of the raw material stream into the product stream, and
said restoration mode comprising restoring the temperature of the bed of fluidized solid particles by passing a restoration stream through the bed of fluidized solid particles to decrease the temperature of the bed of fluidized solid particles in case the temperature of the fluidized solid particles has increased during the production mode or to increase the temperature of the fluidized solid particles in case the temperature of the fluidized solid particles has decreased during the production mode and that
the fluidized solid particles are kept in the same reactor.

The term “stream” as used herein refers to a flow of material that may include gaseous, liquid and/or solid components.

The term “raw material stream” as used herein refers to the total flow of material that is entering the fluidized bed of the present method whilst it is operated in the production mode and that leaves the fluidized bed as the product stream. It should be understood that the material stream may be composed of several streams that become mixed before they enter the fluidized bed or that become admixed whilst they pass through the fluidized bed.

The term “restoration stream” as used herein refers to the total flow of material that is entering the fluidized bed of the present method whilst it is operated in the restoration mode. Also the restoration stream may be composed of several streams that become mixed before they enter the fluidized bed or that become admixed whilst they pass through the fluidized bed. The restoration stream employed in the present invention releases energy or absorbs energy whilst passing through the fluidized bed. This can be achieved, for instance, by employing a restoration stream having a temperature that is different from the temperature of the fluidized solid particles in the bed and allowing heat exchange to occur. The invention also encompasses the use of a restoration stream comprising components that will undergo an endothermic or exothermic chemical reaction in the fluidized bed or a physical phase change, thereby absorbing energy from or releasing energy to the fluidized solid particles.

The terminology “converting a raw material stream into a product stream” as used herein refers to both chemical and physical conversion. Thus, the present invention encompasses a method in which one or more reactants contained in the raw material stream are converted to into different chemical entities. The method also encompasses a process wherein no chemical reactions occur, but wherein, for instance, the physical state of one or more components in the raw material is changed (e.g. from liquid to vapour) or wherein the energy of the raw material stream is altered during its passage through the fluidized bed (e.g. conversion of saturated steam into superheated steam).

The term “production energy” refers to all energy associated with the conversion of the raw material stream into the product stream, and comprises conversion energy such as reaction energy and phase transition energy (e.g. evaporation), and also the energy associated with cooling or heating the raw material stream inside the fluidized bed. During the production mode of the present method the “production energy” is thermally released or absorbed by the fluidized solid particles.

As used herein, a “positive flow” of production energy involves production energy that is released by the fluidized solid particles in the bed in the production mode and a “negative flow” of production energy is production energy that is absorbed by the bed of fluidized solid particles in the production mode. Endothermic chemical reactions are a cause of positive flows of production energy, and exothermic chemical reactions are a cause of negative flows of production energy.

In the present method the chemical composition of the raw material stream and the restoration stream are different. Nonetheless, despite these compositional differences both streams may contain the same components, e.g. water, steam, nitrogen etc.

The present method can be operated in an effective manner by continuously monitoring a parameter that is indicative of the temperature of the fluidized solid particles and by switching from the production mode to the restoration mode and vice versa in dependency of said parameter. According to a particularly preferred embodiment, the production mode is continued until the temperature of the fluidized solid particles has changed to a value where the production mode cannot be carried out favourably, i.e. it has either increased to a preset maximum value or decreased to a preset minimum value and wherein during the subsequent restoration mode the temperature of the fluidized solid particles is restored to a preset target value. It is noted that this embodiment of the invention can be realised by monitoring one or more parameters that are indicative of the temperature of the fluidized solid particles. An example of a parameter that can suitably be used for this purpose is the temperature of the product stream.

In the aforementioned embodiment, the target value of the temperature typically lies within the range of 50-1200° C. More preferably, said target value is in the range of 110-1000° C., most preferably in the range of 300-900° C. The difference between the preset minimum value and the target value or between the preset maximum value and the target value usually lies within the range of 5-400° C. Preferably said difference is in the range of 10-200° C. and most preferably in the range of 20-150° C.

The duration of both the production mode and the restoration mode can vary within wide ranges. Advantageously, the duration of the production mode is 1-200 minutes and the duration of the restoration mode is 0.5-60 minutes. More preferably, the duration of the production mode is 3-60 minutes and the duration of the restoration mode is 1-60 minutes.

In a preferred embodiment of the present method at least a part of the raw material stream is replaced by the restoration stream when the method switches from the production mode to the restoration mode. Typically at least 5 wt. %, more preferably at least 10 wt. % and most preferably at least 20 wt. % of the raw material stream is replaced by the restoration stream when the method switches from production mode to restoration mode.

As explained herein before, the present method can be operated for an indefinite period of time as the method is extremely robust due to the fact that it does not employ complex (sensitive) equipment and because the restoration mode effectively prevents the process from ‘drifting’. Thus, in accordance with another preferred embodiment, the method comprises an uninterrupted sequence of at least 3 production modes and at least 3 restoration modes. Even more preferably, the present method comprises an uninterrupted sequence of 10 cycles, wherein each cycle comprises a single production mode and a single restoration mode.

Due to its robustness, the present method can suitably be operated in a continuous fashion for at least 24 hours, preferably for at least 72 hours.

The present method offers the advantage that it does not require recirculation of externally heated or cooled solid particles comprised in the fluidized bed, nor heat transfer through walls (including pipe walls of heat exchangers) bordering the fluidized bed, to ensure that the temperature of the solid bed particles is maintained at a stationary level. Thus, in accordance with an advantageous embodiment, the method does not rely on energy transfer by means of recirculation of externally heated or cooled solid particles nor by means of heat exchangers placed inside or enclosing the fluidized bed. More particularly, it is preferred that at least 20%, more preferably at least 30% of the production energy is provided or absorbed by the restoration stream. It should be understood that the energy provided or absorbed by the restoration stream includes energy that is released or absorbed by the restoration stream in the fluidized bed as a result of chemical reactions between reactants contained in the restoration stream and/or between matter contained in the fluidized bed and reactants contained in the restoration stream.

The benefits of the present invention are particularly manifest in case the fluidized solid particles remain fluidized in the bed throughout the production mode. According to a particularly preferred embodiment, the fluidized solid particles also remain fluidized in the bed throughout the restoration cycle, meaning that the fluidized solid particles remain fluidized in the bed throughout the cycle comprising a single production mode and a single restoration mode.

The present method may be operated with fluidized solid particles of varying diameter. Typically, the fluidized solid particles have a volume weighted average diameter in the range of 10-5000 μm, preferably in the range of 50-3000 μm and most preferably in the range of 100-2000 μm.

The solid particles employed in the fluidized bed may be inert or they may actually participate in the conversion of the raw material stream into the product stream and/or restoration stream, e.g. by catalysing a chemical reaction. Examples of fluidized solid particles that can suitably be employed in the present method include particles largely consisting of silicate (e.g. sand), heterogeneous catalyst, metal, metal oxide and combinations thereof.

It is preferred that in the present method the restoration stream causes the temperature of the fluidized solid particles to decrease due to energy absorption by the restoration stream and/or due to the occurrence of endothermic chemical reactions or, alternatively, that the restoration stream causes the temperature of the fluidized solid particles to increase due to energy release from the restoration stream and/or due to the occurrence of exothermic chemical reactions.

In a preferred embodiment, the present method concerns a production mode in which the conversion of the raw material stream into the product stream causes a temperature decrease of the fluidized solid particles (i.e. its processing by the fluidized bed requires a positive flow of production energy). Typically, in accordance with this embodiment of the invention, the temperature of the fluidized solid particles decreases by at least 10° C., preferably by at least 20° C. and most preferably by at least 30° C. during the production mode.

In case the production mode involves a positive flow of production energy, it is advantageous to subsequently employ a restoration stream that comprises an energy carrier selected from the group consisting of hot gasses, hot vapours, hot liquids, or reactants that react exothermically under the conditions prevailing in the fluidized bed during the restoration mode and combinations thereof.

In case the restoration mode increases the temperature of the fluidized bed, it can be advantageous if during the restoration mode the fluidized solid particles catalyse an exothermic chemical reaction of reactants contained in the restoration stream.

Another preferred embodiment for increasing the temperature of the fluidized bed is a method wherein, during the restoration mode, an exothermic chemical reaction occurs between reactants contained in the restoration stream and matter that was accumulated in the bed of fluidized solid particles during the production mode. An example of such a matter is carbon. A suitable reactant that can react exothermically with the carbon matter is oxygen.

In a further preferred embodiment of the invention the restoration mode is performed by introducing a combustion medium, such as fuel and/or oxygen or air into the fluidized bed such that a combustion reaction occurs in the fluidized bed.

According to yet another embodiment, the restoration mode is realised by passing a hot gas stream through the fluidized bed. This option is very much preferred under circumstances where a source of hot gas is available.

In the above described embodiments of the present method that are characterized by a positive flow of production energy and, therefore, a temperature increase of the fluidized bed during the restoration mode, preferably at least 30%, more preferably at least 50% and most preferably at least 70% of the production energy is provided by the restoration stream. Even more preferably, the latter energy percentages are generated by exothermic chemical reactions occurring within the fluidized bed concurrent with the passage of the restoration stream.

Examples of processes of a positive flow of production energy that can occur in the fluidized bed during the production mode of the present method include heating of raw material streams, pyrolysis, dehydrogenation, evaporation, steam reforming, devolatilization and combinations thereof. These processes may be carried out using a raw material stream that comprises a raw material selected from the group consisting of biomass, coal, water, steam, C₁-C₁₀ alkanes, waste polymers and more preferably from recycled waste polymers such as from automotive tyres and from bottles, carpets, packaging material, furniture and office equipment, and combinations thereof such as contained in mixed construction and demolition waste, as well as pyrolytic oil such as derived from the above materials.

According to a particularly preferred embodiment, at least 50 wt. % , more preferably at least 70 wt. % and most preferably at least 80 wt. % of the raw material stream consists of the aforementioned raw material.

The term “biomass” as used herein refers to materials, originating from plants and animals, which are suitable for use as starting material for industrial products, including fractions and derivatives of these biological materials. Most commonly, biomass refers to plant matter grown for use as biofuel, but it also includes plant or animal matter used for production of fibres, chemicals or heat. Biomass may also include biodegradable wastes that can be burnt as fuel. The term biomass excludes organic material which has been transformed by geological processes into substances such as coal or petroleum. Examples of said biomass are wood chips, sugar cane bagasse, sugar beet bagasse, straw, hemp grass and the like.

A suitable example of a process of a positive flow of production energy that can be executed during the production mode of the present method is a devolatilization reaction of biomass or coal carried out at an elevated temperature above 600° C. In such a reaction, the biomass or coal reactant is decomposed and devolatilized in the absence of in particular oxidizing materials such as oxygen or air. Where there is no means employed of delivering the associated production energy into the fluidized bed, the temperature prevailing in the fluidized bed decreases down to a level where process conditions inside the fluidized bed and thermal conditions of the solid particles are no longer favourable for the desired devolatilization reaction.

In accordance with another embodiment the production mode comprises the production of superheated steam of 120-150° C. (or any other desired temperature range) and a suitable pressure out of steam of a lower temperature and the same suitable pressure. If the bed material consist of sand particles, and if the production mode is started at a moment in time where the bed temperature equals the upper side of the desired temperature range (e.g. 150° C.), then, with lapse of time the sand particles will cool down until a moment in time when they will no longer be capable of providing sufficient energy to the steam. Subsequently, the process conditions inside the fluidized bed and the temperature of the solid particles can be restored during the restoration mode, for instance, by carrying out a combustion reaction inside the fluidized bed.

In another preferred embodiment, the present method concerns a production mode in which the conversion of the raw material stream into the product stream causes a temperature increase of the fluidized solid particles (i.e. its processing by the fluidized bed requires a negative flow of production energy).

Typically, in accordance with this embodiment of the invention the temperature of the fluidized solid particles increases by at least 10° C., preferably by at least 20° C. and most preferably by at least 30° C. during the production mode.

In an advantageous embodiment of the invention, an exothermic chemical reaction of reactants contained in the raw material stream that is catalyzed by the fluidized solid particles causes the negative flow of production energy during the production mode.

In another preferred embodiment of the production mode with a negative flow of production energy, at least 60%, preferably at least 90% and most preferably at least 95% of the production energy is generated by exothermic chemical reactions occurring within the fluidized bed concurrent with the passage of the raw material stream.

Examples of raw materials that can contribute a negative flow of production energy in the production mode include reactive hydrocarbons, biomass, pyrolytic oil, carbon monoxide, water, steam, carbon, oxygen and combinations thereof. According to a particularly preferred embodiment, at least 50 wt. %, more preferably at least 70 wt. % and most preferably at least 80 wt. % of the raw material stream consists of the aforementioned raw materials. Preferably, the reactive hydrocarbons are selected from the group consisting of saturated and/or unsaturated C₁-C₂₀ alkanes, ketones, aldehydes and combinations thereof.

A variety of exothermic reactions can be executed in the production mode of the present method. Examples of processes having a negative flow of production energy that can occur in the fluidized bed during the production mode of the present method include oxidation, water gas shift reaction and combinations thereof.

In case the production mode yields a negative flow of production energy, energy can suitably be removed from the same fluidized bed during the restoration mode by employing a restoration stream containing one or more components selected from the group consisting of biomass, coal, water, steam, C₁-C₁₀ alkanes, and combinations thereof. Advantageously, at least 50 wt. % , more preferably at least 70 wt. % and most preferably at least 80 wt. % of the restoration stream consists of these components.

An example of such an exothermic reaction that can advantageously occur in the production mode of the present method is the water gas shift reaction. In this reaction CO and H₂O react to form H₂ and CO₂. Typically, the water gas shift reaction is carried out at a temperature of about 700 to 800° C. Where there is no means employed of removing the associated reaction energy from the fluidized bed, the temperature prevailing in the fluidized bed increases up to a level where process conditions inside the fluidized bed and thermal conditions of the solid particles are no longer favourable for the desired water gas shift reaction.

The restoration of the process conditions inside the fluidized bed and of the thermal conditions of the solid particles in the bed can be carried out following several alternatives or combinations thereof. In accordance with one particular alternative, this is achieved by providing a cold gas or vapour flow during the restoration mode. This is an elegant option under circumstances where a source of cold gas or vapour is available and wherein the resulting heated gas or vapour stream is useful for other applications. For instance, if the coolant medium is steam and said steam is gaining superheat during this action, the restoration mode is in itself a productive mode for the generation of superheated steam. Thus, the given restoration mode of the water gas shift reaction is in itself a productive mode for the generation of superheated steam.

For a continuous method for making a product, it is advantageous to use two or more fluidized beds. Accordingly it is possible when using two fluidized beds that in one fluidized bed the production mode takes place and in another fluidized bed the restoration mode. By intermittently switching each fluidized bed from one mode to the other and having both fluidized beds not at the same time in the same production mode or in the same restoration mode, it is possible to carry out the method for making the product in a continuous manner. Under circumstances that the period of time for carrying out the production mode is not substantially the same as the time period for carrying out the restoration mode, it is possible to use different numbers of fluidized beds in the production mode and/or in the restoration mode.

Accordingly, an especially preferred embodiment of the present method employs at least two beds of fluidized solid particles that each are alternately operated in the production mode and the restoration mode, said at least two beds including a first bed that is operated in the production mode and a second bed that is simultaneously operated in the restoration mode, wherein the first bed is switched from the production mode to the restoration mode by diverting at least a part of the raw material stream from the first bed to the second bed and simultaneously the second bed is switched from the restoration mode to the production mode by diverting at least a part of the restoration stream from the second bed to the first bed when (i) the temperature of the fluidized solid particles in the first bed has increased to a preset maximum value or decreased to a preset minimum value or (ii) the temperature of the fluidized solid particles in the second bed has been restored to preset target value.

This particular set-up of the present method is particularly advantageous in case the production mode is endothermic and the restoration mode is exothermic and at least a part of the product stream generated during the production mode in either of the two beds is introduced as reactant into the restoration stream that is fed to the other bed. In a particularly preferred set-up of this embodiment the production mode comprises conversion by steam reforming of a raw material stream comprising water and a hydrocarbon and the restoration mode comprises conversion by water gas-shift reaction of a restoration stream comprising steam and at least a part of the carbon monoxide generated during the production mode. In this particularly advantageous embodiment of the present invention both the production mode and the restoration mode are productive as they both produce hydrogen.

The benefits of the present invention can be realised by operating the method in a manual or automatic fashion. Preferably, the method comprises monitoring the temperature of the fluidized solid particles and automatic switching between the production mode and the restoration mode in dependency of the measured temperature.

In the present method the production mode is advantageously carried out in an adiabatically insulated system, i.e. a system that does not use external heating or cooling.

It will be appreciated that the process of the present invention provides significantly lower investment and maintenance costs per unity of product, due to considerable savings in additional external equipment used in, and reduced control complexity of, prior art processes.

Another aspect of the invention relates to an apparatus for carrying out the method of the invention, comprising at least one vessel holding a bed of fluidizable solid particles, said vessel further comprising:
one or more inlets for a fluidizing stream located at one side of the bed;
one or more outlets located at the opposite side of the bed;
primary switching means for alternately feeding the raw material stream or the restoration stream through the one or more inlets.

Advantageously, the aforementioned apparatus can suitably be used to operate two concurrent processes as described herein before, Accordingly, in a preferred embodiment, the apparatus comprises two or more of vessels holding a bed of fluidizable solid particles, including a first vessel and a second vessel, wherein the primary switching means is capable of redirecting the raw material stream from the first vessels to the second vessel whilst simultaneously redirecting the restoration stream from the second vessel or from another one of the two or more vessels to the first vessel. In a particularly advantageous embodiment of the latter apparatus at least one of the inlets of the first vessel is connected to at least one of the outlets of the second vessel and at least one of the inlets for the second vessel is connected to at least one of the outlets of the first vessel and the apparatus further contains secondary switching means for alternately feeding a stream from the outlet of one vessel to the inlet of the other vessel, wherein the operation of the secondary switching means is synchronized with the operation of the primary switching means.

Although the apparatus may be provided with various inlets, it can be practical that inlets are combined (likewise for the various outlets) and that sources for the various material streams can be switched onto one combined inlet or outlet, and that used restoration streams and ready products may be directed to different utilities from a combined outlet.

To adequately balance a production mode and a restoration mode of one fluidized bed reactor, or of an array of multiple fluidized bed reactors, it can be advantageous to operate sections of a fluidized bed in different fluidization regimes, such as bubbling, slugging or spouting. Advantages associated with this set-up are associated with the energy storage capacity and energy transfer capability of the solid bed particles in the different bed sections. The means for creating different fluidization regimes in distinguished bed sections include but are not limited to: the installation of internal draft tubes or baffles, and the installation and operation of utilities for modulated gas flows that are specifically heated or cooled.

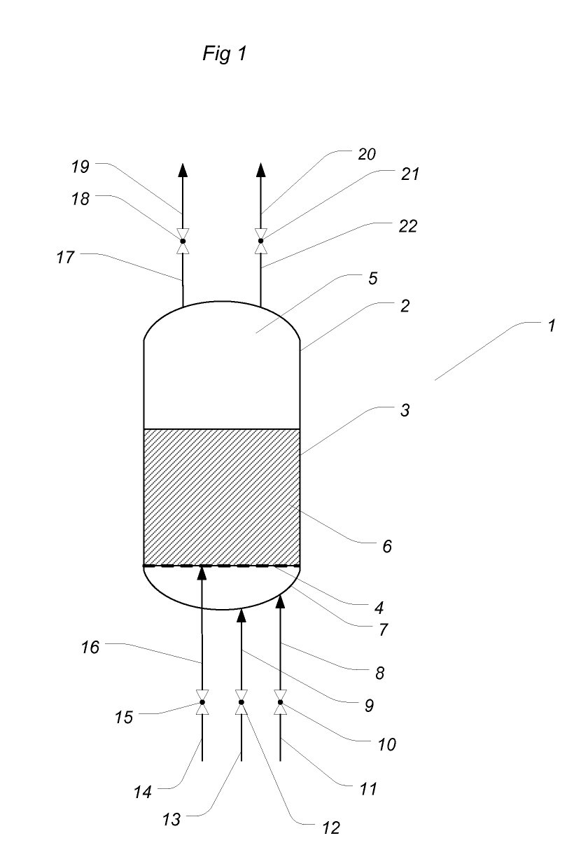

FIG. 1 shows an apparatus 1 by means of which a biomass devolatilization may be carried out. The apparatus 1 comprises a fluidized bed unit 2 having a cylindrical fluidized bed reactor 3 provided at its bottom with a distribution plate 4 and at its top with a freeboard 5 for the separation of solid particles and fluidization medium.

In the reactor 3 is maintained a fluidized bed of sand particles 6 (at average particle size of about 0.4 mm) as is shown by the shaded area.

The fluidized bed 6 is provided with inlet 16, which is connected via a valve 15 with a source of cold biomass 14. A bottom section 7 of the reactor 3 is provided with two inlets 8 and 9. The inlet 8 is connected via a valve 10 with a source of combustion air 11. The inlet 9 is connected via a valve 12 with a source of fuel 13.

The freeboard 5 is provided with two outlets 17 and 22. Outlet 17 is connected via a valve 18 to an outlet for exhaust gas 19. Outlet 22 is connected via valve 21 to an outlet for the devolatilization product 20.

During the restoration mode valves 10 and 12 are open and thus combustion air and fuel are flown into the reactor 3. Exhaust gas 19 leaves the apparatus 1 via the open valve 18. The combustion energy is taken up by the sand particles, thereby are the process conditions reconstituted in the fluidized bed and the solid bed material brought into a temperature condition enabling the provision of process energy for the intended process during the subsequent production mode. After the process conditions have been reconstituted along with the capability of the sand particles to provide process energy, the valves 10, 12 and 18 are closed and the valves 15 and 21 opened. Cold biomass 14 enters the reactor 3 and the devolatization of added biomass takes place within a temperature window of about 650-550° C. Upon continuation of the devolatilization reaction, and as a result of the energy consumption of cold biomass for heating and/or of the chemical reaction (which may be or not be endothermic), the temperature of the fluidized bed 6 is no longer favourable for the desired devolatilization reaction and the sand particles comprising the fluidized bed 6 are no longer capable of providing the process energy at the desired temperature so that the devolatilization reaction will occur at a low rate and/or at a low yield. At such temperature (such as below 550° C.) the reactor 3 is switched to operation in the restoration mode as described above.

FIG. 1 also serves to schematically illustrate a method and apparatus by means of which a dehydrogenation of C₃-C₄ paraffins (alkanes) to their corresponding mono-olefins may be carried out. The dehydrogenation of C₃-C₄ paraffins is an example of an endothermic reaction, which is preferably carried out by using a solid chromium or platinum based catalyst, and at a temperature above 1000° C. to minimize the formation of by-products and to maximize yield. The catalyst solid matter may form part or all of the solid fluidized bed particles. During the production mode, according to the invention, valve 15 is opened to admit the C₃-C₄ paraffins into the fluidized bed reactor 3. Thereby, the solid fluidized bed particles, by their cooling, will provide the process energy for carrying out the production mode, until the temperature of the fluidized bed is no longer favourable for the desired dehydrogenation reaction and the solid particles comprising the fluidized bed are no longer capable of providing the process energy at the desired temperature so that the dehydrogenation reaction will occur at a low rate and/or at a low yield. At such temperature the reactor 3 is switched to operation in the restoration mode as described above. In a preferred embodiment the restoration mode is carried out by the combustion of carbon deposited on the solid bed particles or otherwise present within the fluidized bed as a result of carbon formation during the production mode. The valve 12, otherwise admitting fuel 13 into the fluidized bed reactor 3, may then remain closed during the restoration mode. This intermittent method is an alternative for the continuous so-called Snamprogetti/Yarsintez process described by Domenico Sanfilippo, Franco Buonomo, Giorgio Fusco, Maria Lupieri and Ivano Niracca (Fluidized Bed Reactors For Paraffins Dehydrogenation, Chemical Engineering Science. Vol. 47, No. 9-11, pp. 2313-2318, 1992).

It is noted that at the start of the processes illustrated by FIG. 1, when the fluidized bed is not yet suitably conditioned for operation in production mode, it is possible to apply via inlet 8 or 9 a hot gas or vapour, or alternatively a fuel can be provided via inlet 9 for combustion inside the fluidized bed.

It is noted that the single apparatus 1 comprising one fluidized bed reactor, may be intermittently operated in between a production mode and a restoration mode. The resulting method is then discontinuous in relation to the making of process product. If the making of process product is desired to be continuous then it is possible to use two or more fluidized bed reactors such that at least one fluidized bed reactor is in the production mode and at least one fluidized bed reactor is in the restoration mode. Accordingly, there is a continuous making of process product.

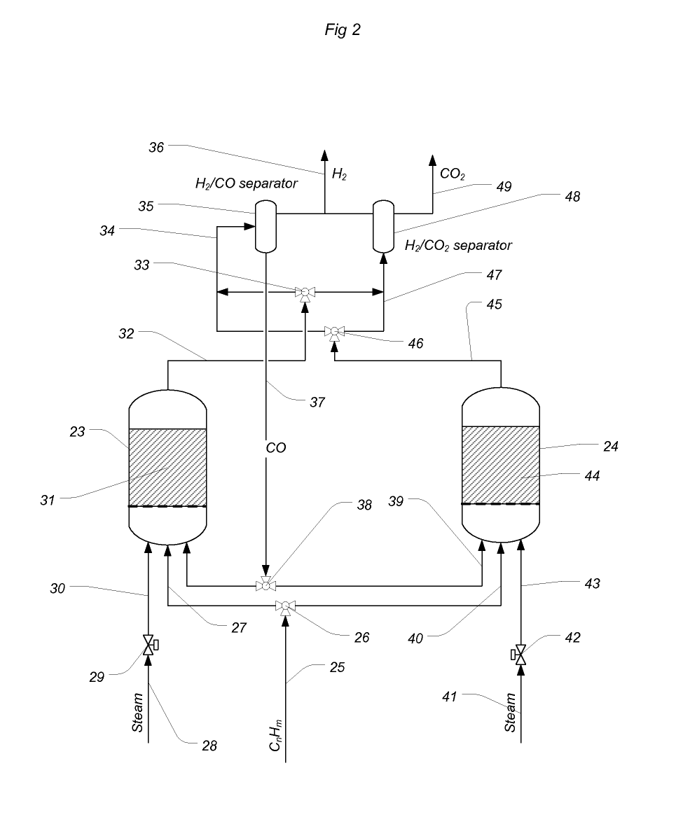

FIG. 2 describes another apparatus according to the invention, comprising two fluidized bed reactors 23 and 24 which are intermittently but not concomitantly operative in different modes. The reactors 23 and 24 have substantially the same construction as described in relation to the fluidized bed reactor 2 in FIG. 1.

In one production mode, hydrocarbons CₙH₂ₙ₊₂ are added via inlet 25 and three-way valve 26 to inlet 27 of the fluidized bed reactor 23. Steam is added via inlet 28, dosage valve 29, and inlet 30 to the fluidized bed reactor 23. The reaction carried out in fluidized bed reactor 23 is steam reforming, which is a conversion of hydrocarbons to carbon monoxide and hydrogen. In the fluidized bed reactor 23 solid particles 31 comprising Ni based catalysts are maintained in a fluidized bed. This catalyst supports the steam reforming reaction, which is carried out at elevated temperatures of 500-1100° C., and which is endothermic. Continued operation of fluidized bed reactor 23 in this production mode, in the absence of specific means for the provision of heat, results in a gradual temperature decrease of the solid fluidized bed particles 31.

Via outlet 32, three-way valve 33 and inlet 34, the reaction product is added to a separation unit 35 in which hydrogen 36 and carbon monoxide 37 are separated, and the hydrogen 36 removed as the product.

The carbon monoxide 37 is fed to the fluidized bed reactor 24 via the three-way valve 38 and inlet 39. Via dosage valve 42 and inlet 43 is supplied steam 41. In the fluidized bed reactor 24 solid particles 44 comprising a catalyst for the water gas shift reaction are maintained in a fluidized mode. These catalyst particles are MnCu and/or Ni based. Suitably, the water gas shift reaction is carried out at a temperature of about 700 to 800° C., which is lower than the preferred temperature for the steam reforming reaction, and the reaction is exothermic. Due to this exothermic character, and in the absence of specific means for cooling, continued operation of fluidized bed reactor 24 in this production mode, results in a gradual temperature increase of the solid fluidized bed particles 44, beyond the upper limit of a suitable reaction temperature range for the water gas shift reaction.

Via outlet 45, three-way valve 46, and inlet 47, the reaction product is fed to a separation unit 48 separating hydrogen 36 from carbon dioxide 49, and the hydrogen 36 removed as the product.

The two fluidized bed reactors 23 and 24 being operated simultaneously, and each of which making another product mixture, the temperature of one fluidized bed reactor gradually increases whereas the temperature of the other gradually decreases up to temperature ranges where the respective productions are no longer effective and/or efficient. According to the invention it is possible to intermittently operate the individual fluidized bed reactors 23 and 24 in subsequent steam reforming and water gas shift reaction modes, in such a manner that when one fluidized bed reactor is operated to carry out steam reforming, the other fluidized bed reactor is operated to carry out the water gas shift reaction. For this operation to be carried out properly, it is required that the three-way valves 26, 33, 38 and 46 are suitably switched, and the dosage valves 29 and 42 suitably controlled. Considering one single fluidized bed reactor of the two reactors 23 and 24, the production mode of hydrogen manufacture by means of the water gas shift reaction, due to the increasing temperature of the solid particles as a result of the reaction's exothermic character, serves as the restoration mode for the making of hydrogen by means of the steam reforming reaction, which is the subsequent production mode carried out in that fluidized bed reactor. In turn, the subsequent production mode of the steam reforming reaction, due to the decreasing temperature of the solid bed particles as a result of that reaction's endothermic character, carried out in the considered fluidized bed reactor, serves as the restoration mode for the making of hydrogen by means of the water gas shift reaction, which is the subsequent production mode carried out in that fluidized bed reactor.

Obviously, the catalytic particle systems used for the steam reforming reaction and for the water gas shift reaction are to be selected such that preferably both catalyst systems could be present in one and the same reactor for carrying out under the appropriate reactions conditions the characteristic catalytic reaction.

For the skilled person it will be routine to decide at what stage a fluidized bed reactor is to be changed from one mode to another. Such decision may be dependent on residing temperatures, reaction enthalpies, heat capacities of solid particles, and those of reconstituting fluidums, and reactants, the reaction rate and the yield of the reaction products, and other items. It will also be routine for the skilled person to design an apparatus consisting of a fluidized bed reactor, or of a combination of multiple fluidized bed reactors acting together, in production modes and restoration modes. Such design may be dependent on the residing temperatures, reaction enthalpies, the heat capacities of solid particles, of reconstituting fluidums, and reactants, the reaction rate and the yield of the reaction products, and other items.

According to a more preferred embodiment of the present invention, biomass or polymer waste is pyrolysed in the production mode while a fuel is combusted with air in the restoration mode.

The invention is further illustrated by means of the following non-limiting examples.

### EXAMPLES

### Example 1

This example illustrates the use of the present method for producing superheated steam of 750-850° C. at atmospheric pressure from saturated steam at the same pressure. The method described utilized a fluid bed reactor as depicted in FIG. 1.

In the restoration mode the fluidized bed reactor was heated to 850° C. by means of coal and combustion air. This raw material stream also acted as fluidization medium. Mode switching was carried out when the temperature of the fluidized bed reached the aforementioned target value of 850° C. by halting the flows of coal and combustion air, and starting a flow of saturated steam which also acted as fluidization medium. The exit of the fluidized bed reactor was simultaneously switched from flue gas to superheated steam (and the steam can be fed into a steam drum). The outlet steam was superheated at a temperature that is essentially equal to the temperature of the fluidized bed.

During the production mode, the temperature of the fluidized bed gradually decreased as a result of heat transfer from the solid bed particles to the steam. When the superheated steam temperature reached the lower end of the desired temperature range (in this example 750° C.), the fluidized bed reactor was switched back to restoration mode and a raw material stream of coal and air was again fed into the bed instead of the flow of saturated steam. Simultaneously, the gas exit of the fluidized bed reactor was connected to a flue gas duct.

### Example 2

This example illustrates the use of the present method for producing pyrolytic oil and vapour from biomass. Again, the method utilized a fluid bed reactor as depicted in FIG. 1. The fluidized bed consisted of sand particles (average diameter=0.5 mm).

In the restoration mode the fluidized bed reactor was heated to 540° C. by combusting natural gas and air inside the fluidized bed. This mixture of raw materials and the resulting combustion products also acted as fluidization medium. Mode switching was carried out when the temperature of the fluidized bed reached the target value by halting the raw material flows of natural gas and combustion air, and starting a flow of inert gas (nitrogen) through the bed to bring it into a regime of minimum fluidization. In addition to nitrogen, the raw material stream contained cold biomass particles that had been processed such that they properly mixed with the sand under the conditions of the fluidization regime that resulted from the pyrolysis reaction. The biomass had been ground to a particle size of less than 2 mm and dried to a moisture content of 0% to ensure optimum intra-biomass heat transfer for the pyrolysis reaction to occur and to minimize energy usage for water evaporation.

During the production mode, the temperature of the fluidized bed decreased as a result of the endothermic pyrolysis of the biomass and of its heating to the reactor temperature. When the fluidized bed reached the lower end of the desired temperature range (of 460° C.), the fluidized bed reactor was switched back to restoration mode.

### Example 3

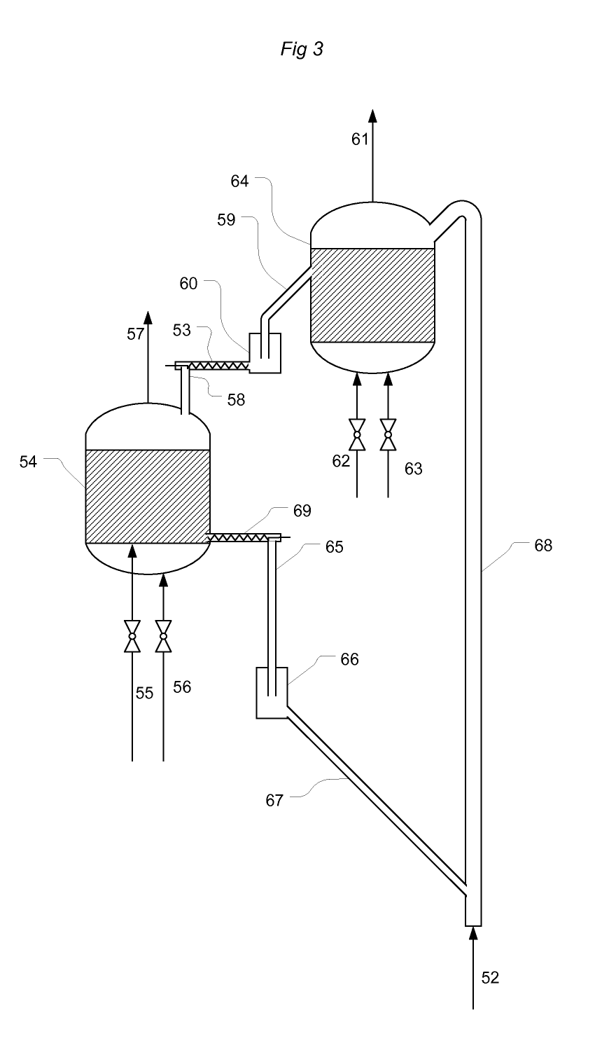

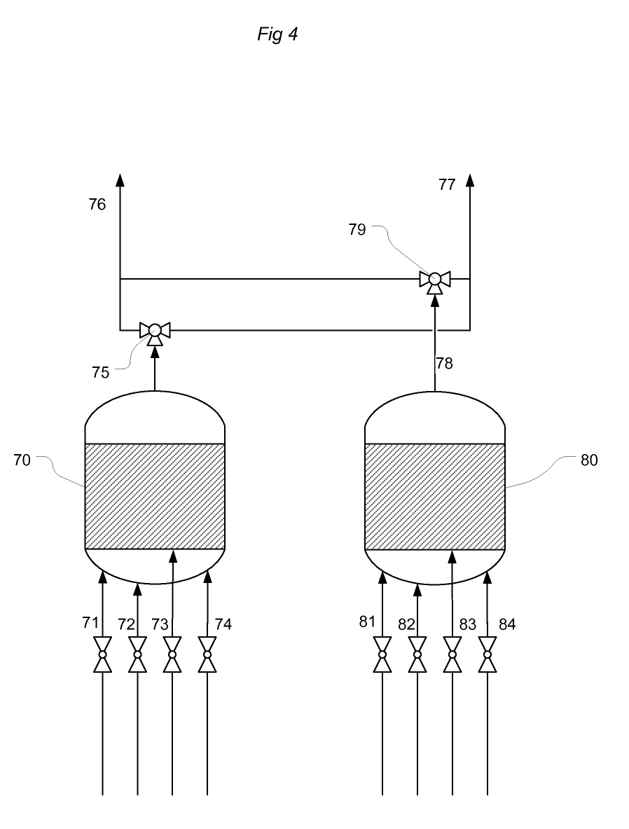

FIGS. 3 and 4 show two configurations of the same processes carried out in fluidized bed reactors. FIG. 3 is according to existing art by means of stationary fluidized bed processes, and FIG. 4 is according to the invention by means of intermittently operated fluidized beds. The primary process is pyrolytic liquefaction of biomass and the energy for that process is provided by means of a combustion reaction. The pyrolytic liquefaction reaction can be favourably carried out inside a fluidized bed of hot sand particles in the absence of air, and within a range of suitable temperatures that is at least 80° C. wide, e.g. between 460 and 540° C. which can be derived from “Fast pyrolysis of sweet sorghum and sweet sorghum bagasse”, Journal of Analytical and Applied Pyrolysis, Volume 46, Issue 1, June 1998, Pages 15-29, Jan Piskorz, Piotr Majerski, Desmond Radlein, Donald S. Scott, A. V. Bridgwater, in particular page 24, FIG. 6.

In FIG. 3, Reactor 54 is the pyrolytic liquefaction reactor. It is fed with biomass at ambient temperature as the raw material via line 55, and an inert gas such as nitrogen is provided as a fluidization medium via line 56. (Alternative gases that may serve this purpose are non-condensed process gases.) Inside Reactor 54 the raw material is heated to the bed temperature of 480° C. and it decomposes into product vapour, the temperature of which equals the bed temperature. The product vapour is removed from Reactor 54 via line 57. The process consumes energy, and this is supplied by a flow of heated sand from Reactor 64, e.g. at a temperature of 800° C., which is essentially higher than the desired process temperature of Reactor 54 (here 480° C.). This flow of heated sand is enabled by gravity, and a number of devices including a Sand Supply Pipe 58, a Sand Extraction Screw acting as flow regulator 53, a Sand Lock 60, and a Stand Pipe 59. These devices are required because it is essential to control the flow of heated sand while avoiding the mixing of reactants and regeneration gases. Inside Reactor 64 a combustion reaction is carried out at the desired sand supply temperature. To this end fuel and combustion air are provided. In some cases all or part of the fuel may already be present in the form of charcoal contained inside the relatively cool sand that is fed into Reactor 64 to balance the sand flow into Reactor 54. The sand taken from Reactor 64 to supply energy to Reactor 54 is balanced by a feed of sand into Reactor 64 that is provided by means of a riser (driven by air through line 52) that transfers sand taken from Reactor 54. In some cases this riser may be a belt conveyor or a bucket elevator, or another mechanical device. Additional devices needed to enable a transfer of sand from Reactor 54 to Reactor 64 include: a frequency controlled Extraction Screw 69 or another mechanical means of solids flow regulation (such as a temperature resistant Pinch Valve), a Stand Pipe 65, a Sand Lock 66 and a Sand Transfer Pipe 67. These devices are required because it is essential to control the flow of heated sand while avoiding the mixing of reactants and regeneration gases. The temperature of the sand removed from Reactor 54 is equal to the temperature of the fluidized bed inside Reactor 54. The process is carried out continuously.

In FIG. 4, each of the Reactors 70 and 80 acts as a pyrolytic liquefaction Reactor and a combustion reactor, however not at the same moment in time, but rather intermittently. In this specific case the two reactors are operated in counter phase, i.e. such that if one is operated in a pyrolytic liquefaction mode that is limited in time, the other is operated in a combustion mode that is limited by the same time interval, and after the time interval has expired the respective operational modes are being switched from pyrolytic liquefaction to combustion and vice versa. Take, for the sake of argument, a moment in time at which Reactor 70 is in the mode of pyrolytic liquefaction and Reactor 80 in the mode of combustion. At that moment, Reactor 70 is fed with biomass at ambient temperature as the raw material via line 73, and an inert gas such as nitrogen is provided as a fluidization medium via line 74. Alternative gases that may serve this purpose are non-condensed process gases. Inside Reactor 70 the raw material is heated to the bed temperature and it decomposes into product vapour, the temperature of which equals the bed temperature. The product vapour is removed from Reactor 70 via valve 75 and line 77. The process consumes energy, and this is supplied by the fluidized bed inside Reactor 70. While providing the energy, the fluidized bed inside Reactor 70 cools down. This process is continued until the temperature of the fluidized bed inside Reactor 70 reaches a temperature that is too low for maintaining a favourable pyrolytic liquefaction reaction. At that moment the operational mode of Reactor 70 is switched from pyrolytic liquefaction to combustion. To this end, the feed of raw material is halted, and fuel and combustion air are provided to Reactor 70 via lines 71 and 72 respectively. In some cases all or part of the fuel may already be present in the form of charcoal remaining as a residue inside Reactor 70. As a result of combustion, the temperature conditions of the fluidized bed inside Reactor 70 are restored, and the intermittent cycle of pyrolytic liquefaction followed by combustion is repeated. Operation of Reactor 80 in counter-phase to Reactor 70 enables a continuous productive process. The actually prevailing mode duration is dependent upon the design parameters such as production capacity and bed size. The mode of pyrolytic liquefaction, which is the primary process objective, may e.g. last 30 minutes, and the restoration mode of combustion may last 5 minutes.

In below table the two systems are compared.

| Stationary fluid bed with external | |
|---|---|
| solids circulation for pyrolytic | Intermittent fluid bed for pyrolytic |
| liquefaction of biomass | liquefaction of biomass |
| In addition to the major process reactor, | One reactor is sufficient for carrying out |
| at least one other reactor is required to | the process of the invention, but to |
| reconstitute the bed solids. | enable the comparison of a continuous |
| | process two reactors were used in |
| | Example 3. |
| Devices enabling external solids | External solids circulation is omitted. |
| circulation are required. | On/off gas-flow valves are required to |
| While allowing the flow of solids, those | switch the process. |
| devices should prevent undesired flows | Those valves should be resistant to the |
| of gases (back-mixing, forward mixing). | prevailing temperatures (460-540° C.). |
| Those devices should be resistant to | |
| high temperature, higher than in the | |
| case of an intermittent fluid bed. | |
| Those devices should be resistant to wear. | |
| Those devices need temperature and flow controls. | |
| Those devices need operational power | |
| (e.g. for a blower inducing a riser). | |
| The process conditions of the major | The process conditions of the major |
| process can be fixed at set-point to | process oscillate around an optimum. |
| optimize production. | |

It will be appreciated from said table that the concept of an intermittent fluid bed for pyrolytic liquefaction of biomass bears a number of advantages over a stationary fluid bed with external solids circulation. Those advantages are in terms of less control complexity, less reactors, less solids transfer devices, less exposure to wear, less operational power. It is reasonable to expect that the intermittent fluid bed for pyrolytic liquefaction of biomass is twice less costly than the stationary fluid bed with external solids circulation, both in terms of investment (equipment) and operational costs (power and maintenance). A disadvantage of an intermittent fluid bed for pyrolytic liquefaction of biomass is the oscillation of process conditions, which may result into a lower product yield as a ratio of raw material input. The productivity reduction could be as much as 5%.

### Example 4

A pyrolysis process of polyethylene waste according to the present invention in one fluidized bed reactor is operated intermittently within a temperature range of from 290° C. to 340° C. The production mode (pyrolysis) starts at 340° C. and the fluidized bed is allowed to cool until a temperature of 290° C. has been reached. As from that moment the fluidized bed is re-heated by combusting the coal formed inside the bed during the production mode (pyrolysis) by feeding air and optionally an additional fuel (restoration mode), until the bed reaches the starting temperature of 340° C. again. The cycle can be repeated as long as required and the fluidized solid particles remain inside the same reactor. The product yield appears to vary within acceptable ranges.

1. A method of converting a raw material stream into a product stream by passing said raw material stream through a bed of fluidized solid particles and allowing heat exchange to occur between the bed of fluidized solid particles and the raw material stream, said method being characterized in that it alternately employs a production mode and a restoration mode to control the temperature of the fluidized solid particles:
said production mode comprising passing the raw material stream through the bed of fluidized solid particles and allowing the temperature of the fluidized solid particles to decrease or to increase as a result of the production energy associated with the conversion of the raw material stream into the product stream; and
said restoration mode comprising restoring the temperature of the bed of fluidized solid particles by passing a restoration stream through the bed of fluidized solid particles to decrease the temperature of the fluidized solid particles in case the temperature of the fluidized solid particles has increased during the production mode or to increase the temperature of the fluidized solid particles in case the temperature of the fluidized solid particles has decreased during the production mode;
and that the fluidized solid particles are kept in the same reactor.

2. Method according to claim 1, wherein the production mode is continued until the temperature of the fluidized solid particles has either increased to a preset maximum value or decreased to a preset minimum value and wherein during the subsequent restoration mode the temperature of the fluidized solid particles is restored to a preset target value.

3. Method according to claim 1, wherein the duration of the production mode is 1-200 minutes and the duration of the restoration mode is 0.5-60 minutes.

4. Method according to claim 1, wherein the method comprises an uninterrupted sequence of at least 3 production modes and at least 3 restoration modes.

5. Method according to claim 1, wherein during the production mode an endothermic chemical reaction of reactants contained in the raw material stream occurs in the fluidized bed

6. Method according to claim 1, wherein at least 20%, preferably at least 30% of the production energy is provided by the restoration stream.

7. Method according to claim 1, wherein the temperature of the fluidized solid particles decreases by at least 10° C., preferably by at least 20° C. and most preferably by at least 30° C. during the production mode.

8. Method according to claim 5, wherein the restoration stream comprises an energy carrier selected from the group consisting of hot gasses, hot vapours, hot liquids, or reactants that react exothermically under the conditions prevailing in the bed of fluidized solid particles during the restoration mode and combinations thereof.

9. Method according to claim 5, wherein during the restoration mode the fluidized solid particles catalyse an exothermic chemical reaction of reactants contained in the restoration stream.

10. Method according to claim 5, wherein during the restoration mode an exothermic chemical reaction occurs between reactants contained in the restoration stream and matter that was accumulated in the bed of fluidized solid particles during the production mode.

11. Method according to claim 1, wherein the temperature of the fluidized solid particles in the bed increases by at least 10° C., preferably by at least 20° C. and most preferably by at least 30° C. during the production mode.

12. Method according to claim 1, wherein during the production mode an exothermic chemical reaction of reactants contained in the raw material stream occurs in the fluidized bed.

13. Method according to claim 11, wherein the restoration stream comprises an energy carrier selected from the group consisting of cold gasses, cold vapours, cold liquids, or reactants that react endothermically under the conditions prevailing in the bed of fluidized solid particles during the restoration mode and combinations thereof.

14. Method according to claim 11, wherein during the restoration mode the fluidized solid particles catalyse an endothermic chemical reaction of reactants contained in the restoration stream.

15. Method according to claim 1, wherein the method employs at least two beds of fluidized solid particles that each are alternately operated in the production mode and the restoration mode, said at least two beds including a first bed that is operated in the production mode and a second bed that is simultaneously operated in the restoration mode, wherein the first bed is switched from the production mode to the restoration mode by diverting at least a part of the raw material stream from the first bed to the second bed and simultaneously the second bed is switched from the restoration mode to the production mode by diverting at least a part of the restoration stream from the second bed to the first bed when (i) the temperature of the fluidized solid particles in the first bed has increased to a preset maximum value or decreased to a preset minimum value or (ii) the temperature of the fluidized solid particles in the second bed has been restored to preset target value.

16. Method according to claim 15, wherein the production mode is endothermic and the restoration mode is exothermic and wherein at least a part of the product stream generated during the production mode in either of the two beds is introduced as reactant into the restoration stream that is fed to the other bed.

17. Method according to claim 16, wherein the production mode comprises conversion by steam reforming of a raw material stream comprising water and a hydrocarbon and wherein the restoration mode comprises conversion by water gas-shift reaction of a restoration stream comprising steam and at least a part of the carbon monoxide generated during the production mode.

18. Method according to claim 1, wherein biomass or polymer waste is pyrolysed in the production mode, while a fuel and preferably natural gas is combusted with air in the restoration mode.

19. An apparatus for carrying out a method as defined in claim 1, said apparatus comprising at least one vessel holding a bed of fluidizable solid particles, said vessel further comprising:
one or more inlets for a fluidizing stream located at one side of the bed;
one or more outlets located at the opposite side of the bed;
primary switching means for alternately feeding the raw material stream or the restoration stream through the one or more inlets.

20. Apparatus according to claim 19, said apparatus comprising two or more of the vessels holding a bed of fluidizable solid particles, including a first vessel and a second vessel, wherein the primary switching means is capable of redirecting the raw material stream from the first vessels to the second vessel whilst simultaneously redirecting the restoration stream from the second vessel or from another one of the two or more vessels to the first vessel.

21. Apparatus according to claim 20, wherein at least one of the inlets of the first vessel is connected to at least one of the outlets of the second vessel and wherein at least one of the inlets for the second vessel is connected to at least one of the outlets of the first vessel and wherein the apparatus further contains secondary switching means for alternately feeding a stream from the outlet of one vessel to the inlet of the other vessel, wherein the operation of the secondary switching means is synchronized with the operation of the primary switching means.
